(19) United States
(12) Patent Application Publication
(10) Pub. No.: US 2012/0101565 A1
(43) Pub. Date: Apr. 26, 2012

(54) BIOABSORBABLE STENT HAVING RADIOPACITY

(75) Inventor: Jonathan S. Stinson, Plymouth, MN (US)

(73) Assignee: BOSTON SCIENTIFIC SCIMED, INC., Maple Grove, MN (US)

(21) Appl. No.: 13/238,094

(22) Filed: Sep. 21, 2011

Related U.S. Application Data
(60) Provisional application No. 61/406,231, filed on Oct. 25, 2010.

(51) Int. Cl. A61F 2/82

(52) U.S. Cl. 623/1.16; 623/1.34

(57) ABSTRACT

A radially expandable stent and methods of making the same, the stent made entirely of a bioabsorbable metal, the stent having a portion of increased radiopacity, wherein the portion of increased radiopacity has one or more of the following characteristics:

i. the wall thickness of the stent in the portion of increased radiopacity exceeds the wall thickness of the wall immediately adjacent thereto by at least 0.0010″;

ii. the width of the stent in the portion of increased radiopacity exceeds the width of the stent immediately adjacent thereto by at least 0.0005″.

### CROSS-REFERENCE TO RELATED APPLICATIONS

This application is claims priority to US Patent Provisional Application No. 61/406,231 filed Oct. 25, 2010, the entire contents of which are hereby incorporated herein by reference.

### BACKGROUND OF THE INVENTION

The disclosed invention relates generally to a medical device and more particularly to a bioabsorbable stent

Intraluminal stents are typically inserted or implanted into a body lumen, for example, a coronary artery, after a procedure such as percutaneous transluminal coronary angioplasty. Such stents are used to maintain the patency of a body lumen by supporting the walls of the lumen and preventing abrupt reclosure or collapse thereof. These stents can also be provided with one or more therapeutic agents adapted to be locally released from the stent at the site of implantation. In the case of a coronary stent, the stent can be adapted to provide release of, for example, an antithrombotic agent to inhibit clotting or an antiproliferative agent to inhibit smooth muscle cell proliferation, i.e., neointimal hyperplasia, which is believed to be a significant factor leading to re-narrowing or restenosis of the blood vessel after implantation of the stent.

Metallic radially expandable stents such as those formed from stainless steel or Nitinol (NiTi) are desirable because of the superior strength and flexibility. One example of a radially expandable stent formed from stainless steel or Nitinol, for example, is shown in

FIG. 1. This stent is disclosed in commonly assigned U.S. Pat. No. 6,818,014 incorporated by reference herein in its entirety.

However, metallic stents can cause complications such as thrombosis and neointimal hyperplasia. Thus, physicians are becoming increasingly interested in bioabsorbable stents rather than metallic stents that are left in the body permanently.

Recently, there has been significant interest in the use of bioabsorbable stents formed from iron. Iron has similar mechanical properties to stainless steel which has superior strength and flexibility which makes the stents easy to deliver through the patient's vasculature.

However, the radiopacity of iron is not sufficient to allow thin wall stents to be readily visible via fluoroscopic techniques which are often used for placement and for follow up visualization of the implanted stent. Attaching conventional noble metal markers such as gold or titanium renders the stent not completely bioabsobable as desired.

There remains a need in the art for a bioabsorbable stent with sufficient fluoroscopic visibility.

### SUMMARY OF THE INVENTION

In some embodiments, the present invention relates to a radially expandable stent and methods of making the same, the stent made entirely of a bioabsorbable metal, the stent having an outer wall surface and an inner wall surface and a wall extending therebetween, the wall characterized by a wall thickness as measure in a radial direction between the outer and inner wall surfaces, the wall comprising a plurality of struts and connectors which are interconnected and which define openings in the wall, each strut and each connector having two opposing sides extending between the outer and inner wall surfaces and having a length, and a width everywhere along its length, the stent having a portion of increased radiopacity, wherein the portion of increased radiopacity has one or more of the following characteristics:

i. the wall thickness of the stent in the portion of increased radiopacity exceeds the wall thickness of the wall immediately adjacent thereto by at least 0.0010″;

ii. the width of the stent in the portion of increased radiopacity exceeds the width of the stent immediately adjacent thereto by at least 0.0005″.

In some embodiments, the present invention the present invention is directed to a radially expandable stent, the stent formed entirely from a bioabsorbable metal, the stent comprising a plurality of serpentine bands interconnected by connectors, each serpentine band comprising a plurality of struts characterized by a length and interconnected by curved end portions which define the length of the struts, wherein the struts are folded along a portion of the length, the folded portion comprising increased radiopacity.

In some embodiments, the stent is formed entirely of a bioabsorbable iron.

In some embodiment, the present invention is directed to a method of increasing the radiopacity of a bioabsorbable metal stent, the method including providing a tubular stent preform formed from a bioabsorbable metal, cutting a strut pattern in the tubular stent preform, partially electropolishing said tubular stent preform, focally depositing said bioabsorbable metal on portions of the tubular stent preform to form portions of increased radiopacity and electropolishing said tubular stent preform,

wherein the portions of increased radiopacity and have one or more of the following characteristics:

i. the wall thickness of the stent in the masked portion of increased radiopacity exceeds the wall thickness of the wall immediately adjacent thereto by at least 0.0010″;

ii. the width of the stent in the masked portion of increased radiopacity exceeds the width of the stent immediately adjacent thereto by at least 0.0005″.

In some embodiments, the present invention relates to a method of increasing the radiopacity of a bioabsorbable metal stent, the method including providing a tubular stent preform formed from a bioabsorbable metal, cutting a strut pattern in the tubular stent preform, partially electropolishing the tubular stent preform, focally depositing said bioabsorbable metal on portions of the tubular stent preform to form portions of increased radiopacity and electropolishing said tubular stent preform, wherein the portions of increased radiopacity and have one or more of the following characteristics:

i. the wall thickness of the stent in the masked portion of increased radiopacity exceeds the wall thickness of the wall immediately adjacent thereto by at least 0.0010″;

ii. the width of the stent in the masked portion of increased radiopacity exceeds the width of the stent immediately adjacent thereto by at least 0.0005″.

These and other aspects, embodiments and advantages of the present disclosure will become immediately apparent to those of ordinary skill in the art upon review of the Detailed Description and Claims to follow.

### BRIEF DESCRIPTION OF THE DRAWINGS

FIG. 1 is a fragmentary flat view of a radially expandable stent in an unexpanded state.

FIG. 2 is a flat view of the outer surface of a single strut.

FIG. 3 is a side view of the single strut taken at section 3 in FIG. 2 wherein the strut is shown having increased thickness.

FIG. 4 is a flat view of a portion of a stent illustrating a plurality of interconnected struts similar to those shown in FIGS. 2 and 3.

FIG. 5 is a radial cross-section of a stent illustrating a plurality of struts similar to those shown in FIGS. 2-4.

FIG. 6 is a side view of an alternate embodiment of a single strut wherein the strut has no increased thickness but has increased width as shown in FIG. 5.

FIG. 7 is a flat view of the outer surface of the strut shown in FIG. 4 wherein a portion of the strut has an increased width.

FIG. 8 is a flat view of a portion of a stent illustrating a plurality of interconnected struts similar to those shown in FIGS. 6 and 7.

FIG. 9 is a radial cross-section of a stent illustrating a plurality of struts similar to those shown in FIGS. 6-8.

FIG. 10 is a side view of the outer surface of a single strut in an alternative embodiment.

FIG. 11 is a flat view of the outer surface of the strut shown in FIG. 6 wherein the curved end portions that interconnect adjacent parallel struts have increased width.

FIG. 12 is a flat view of a portion of a stent illustrating a plurality of interconnected struts similar to those shown in FIGS. 10 and 11.

FIG. 13 is a radial cross-section of a stent illustrating a plurality of struts similar to those shown in FIGS. 10-12.

FIG. 14 is a flat view of the outer surface of a single strut in an alternative embodiment.

FIG. 15 is a side view of the strut taken at section 9 in FIG. 8 wherein a portion of the strut has increased thickness.

FIG. 16 is a flat view of a portion of a stent illustrating a plurality of struts similar to those shown in FIGS. 14 and 15.

FIG. 17 is a radial cross-section of a stent illustrating a plurality of struts similar to those shown in FIGS. 14-16.

FIG. 18 is a side view of an elongated strut.

FIG. 19 is a side view of the elongated strut shown in FIG. 10 after folding a portion of the strut.

FIGS. 20a-20d are side views of a single strut illustrating a process for making a stent according to the invention.

FIGS. 21a-21d are side views of a single strut illustrating an alternative process for making a stent according to the invention.

### DETAILED DESCRIPTION OF THE INVENTION

While embodiments of the present disclosure may take many forms, there are described in detail herein specific embodiments of the present disclosure. This description is an exemplification of the principles of the present disclosure and is not intended to limit the disclosure to the particular embodiments illustrated.

The present invention relates to bioabsorbable radially expandable stents. As used herein, bioabsorbable stents shall refer to those that can be advantageously eliminated from body lumens after a predetermined, clinically appropriate period of time, for example, after the traumatized tissues of the lumen have healed and a stent is no longer needed to maintain the integrity of the lumen. The conventional bioabsorbable materials from which such stents are made are selected to resorb or degrade over time, thereby eliminating the need for subsequent surgical procedures to remove the stent from the body lumen if problems arise.

Suitably, the bioabsorbable stents disclosed herein lose between 0 to about 30% of their original radial force in the first 6 months after implantation, and then thereafter disintegrate into pieces (100% strength loss) in about 12 to about 36 months after implantation. Suitably, all of the original stent material is converted to the biocompatible degradation product or to chemical species already present in the body in 12-48 months after implantation.

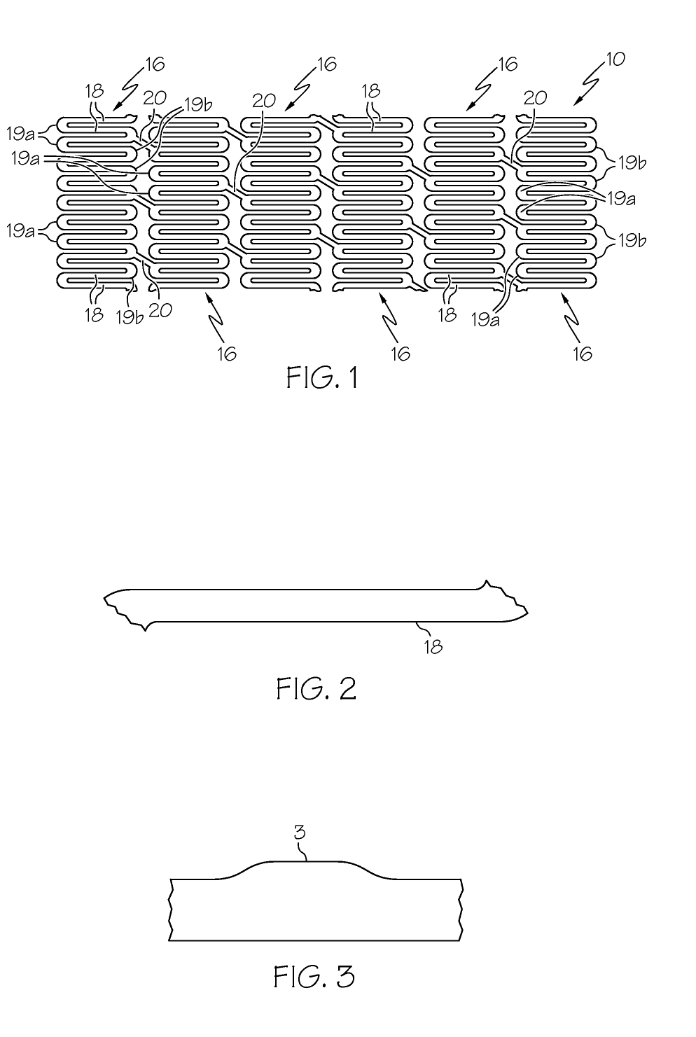

One example of a radially expandable stent construction is shown in FIG. 1 disclosed in commonly assigned U.S. Pat. No. 6,818,014, the entire content of which is incorporated by reference herein. Stent 10 is formed from a plurality of adjacent serpentine segments 16 connected by connector elements 20. Each serpentine band is made up of a plurality of parallel struts 18 interconnected by curved end portions 19a, 19b.

While the connector elements 20 in this embodiment are straight, curved connector elements can also be employed. Furthermore, while connector elements are shown extending from outer curved end portions 19a, 19b, they could also extend from the inner surface of troughs of the serpentine bands 16 (embodiment not shown) rather than the outer curved end portions 19a, 19b. This is only one example of a radially expandable stent and is not intended as a limitation on the scope of the present invention. Those of ordinary skill in the art are well aware of various stent constructions.

Suitably, the bioabsorbable stents disclosed herein have an average wall thickness that is less than current commercially available stainless steel stents. For example, strut thickness may range from 0.0020″ to 0.0055″ (about 50 microns to about 140 microns) and strut width from 0.0025″ to 0.0060″ (63.5 microns to about 152 microns). The more highly radiopaque areas of the strut may range in thickness from 0.0035″ to 0.0065″ (about 89 microns to about 165 microns) or in width from 0.0030″ to 0.0070″ (about 76 microns to about 178 microns).

It is desirable to provide these bioabsorbable stents with portions having an increased wall thickness of at least about 0.0010″ (about 25 microns) or 0.0015″ (about 38 microns) or increased width of at least about 0.0005″ or 0.0010 relative to the stent wall immediately adjacent thereto so that the stents have sufficient radiopacity for visibility using fluoroscopic techniques.

Preserving portions of the stent that have a thinner wall thickness increases the flexibility of the stent and increases the rate at which is absorbed.

Adding radiopaque markers formed from platinum, gold, palladium, iridium and so forth would result in stents that are not completely bioabsorbable as desired herein.

Each of the following figures illustrates a variety of alternate embodiments wherein at least a portion of the stent has an increased width of at least about 0.0005″ (about 12 microns) or an increased thickness of at least about 0.0010″ (about 25 microns) relative to the stent wall immediately adjacent thereto. These various embodiments are intended for illustrative purposes only, and not as a limitation on the scope of the present invention.

The areas of increased thickness or width may be included on every strut 18 of every serpentine band 16, on every strut 18 of every other serpentine band 16, or on every strut of the proximal band, distal band and middle band or a combination thereof. Of course, this pattern can be varies so that every other strut 18, every third strut 18 and so forth of the band 16 might include the areas of increased thickness or width. In a preferred embodiment, the all the struts 18 of the proximal, distal and middle bands 16 have areas of increased thickness or width. Of course, connectors 20 could also include the portions of increased thickness.

FIG. 2 is a flat view of the outer surface of a single strut 18 according to the invention. Section 3 in FIG. 2 illustrates a portion of the strut 18 having a portion 22 having increased wall thickness of at least about 0.0010″ as related to the wall immediately adjacent thereto for increased radiopacity in this portion.

FIG. 3 is a side view taken at section 3 in FIG. 2 to illustrate the increased thickness of a portion of the stent strut 18.

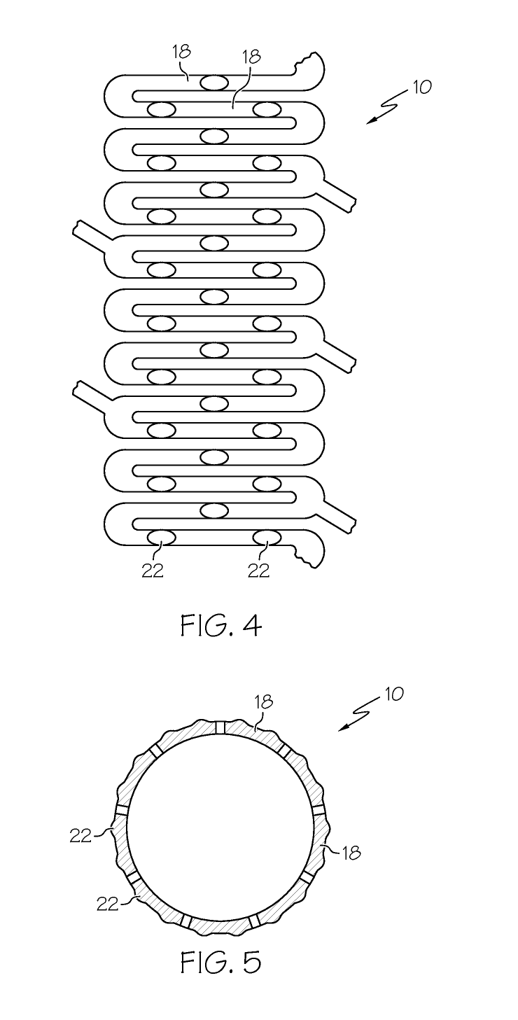

FIG. 4 is a flat view of a portion of a stent 10 wherein every strut 18 has portions 22 of increased thickness. In this case, the outer diameter only is shown with portions 22 of increased thickness.

FIG. 5 is a radial cross-section illustrating a stent 10 having struts 18 as in FIGS. 2-4.

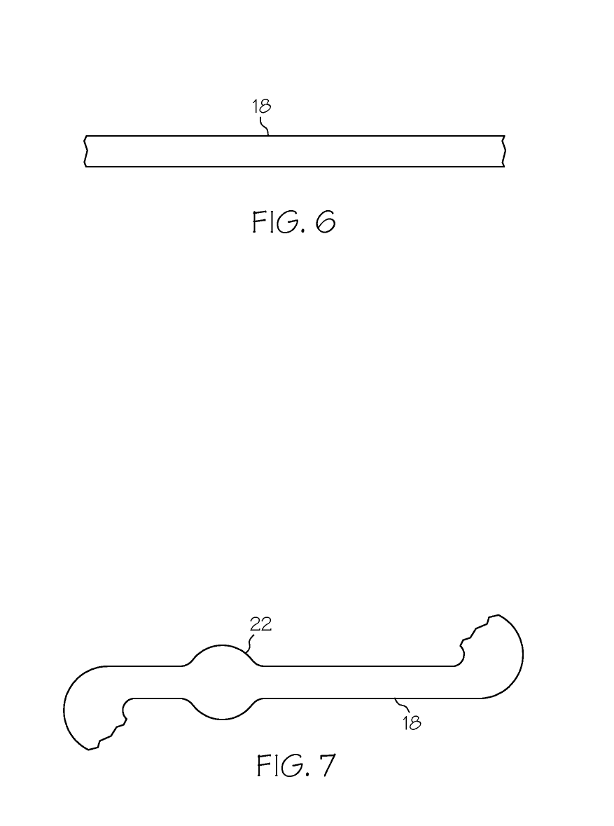

FIG. 6 is a flat view of the outer surface of a single strut 18 wherein a portion of the strut 22 has an increased width of at least about 0.0005″ (about 12.5 microns). FIG. 7 is a flat view of the outer surface of strut 18 shown in FIG. 6.

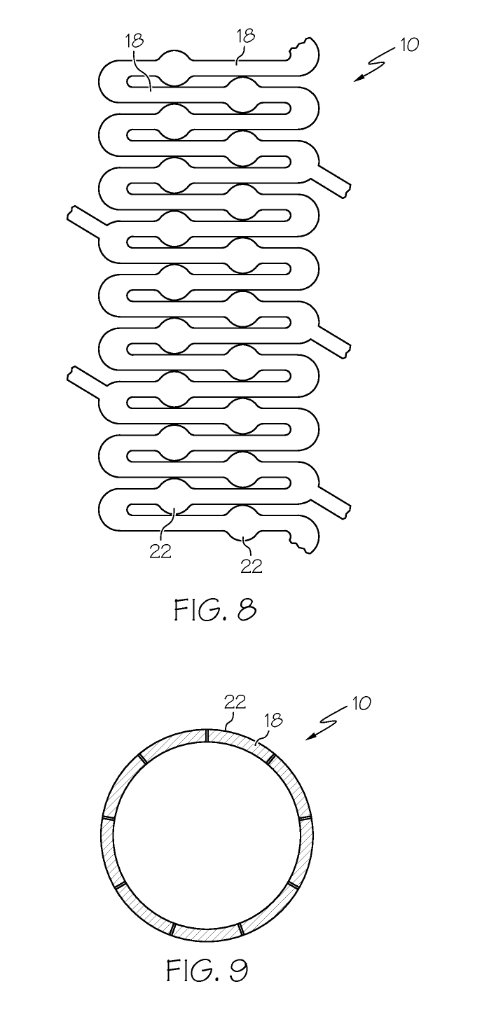

FIG. 8 is a flat view of a portion of a stent 10 wherein every strut 18 is shown having portions 22 of increased width. FIG. 9 is a radial cross-section illustrating stent 10 having struts 18 similar to those shown in FIGS. 6-8.

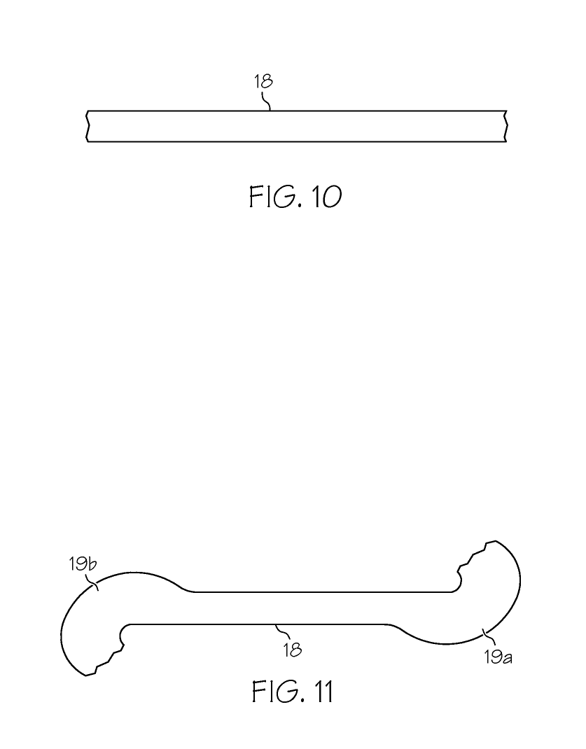

FIG. 10 is a side view of a single strut in another alternative embodiment of the stent according to the invention. In this embodiment, strut 18 includes curved end portions 19a, 19b having an increased width of at least about 0.0005″ (about 25 microns) as shown in a flat view of the outer surface in FIG. 11.

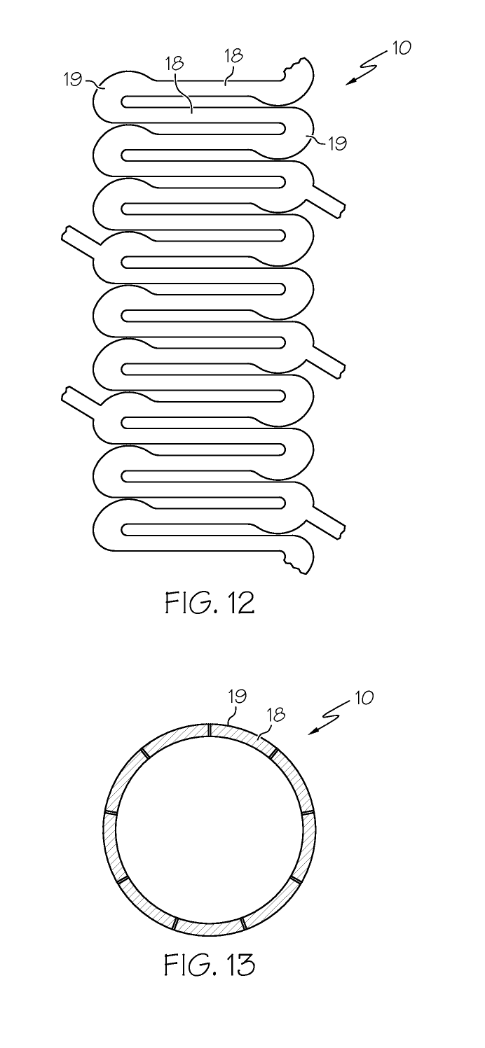

FIG. 12 is a flat view of a portion of a stent 10 having interconnected struts 12 wherein the curved end portions 19 have increased width.

FIG. 13 is a radial cross-section of a stent 10 similar to that shown in FIG. 12

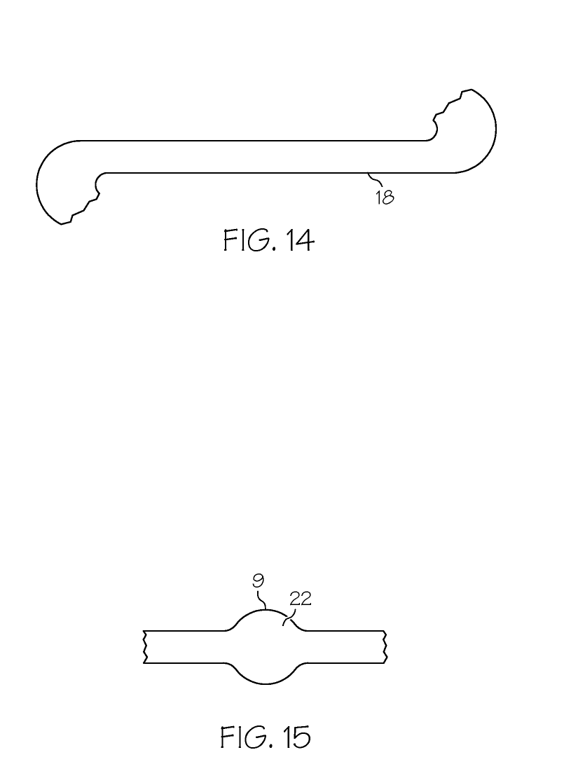

FIG. 14 is a flat view of the outer surface of a single strut 18 in yet another embodiment of the stent according to the invention. In this embodiment, strut 18 has a portion 22 having an increased thickness taken at section 9 in FIG. 14. FIG. 15 is a side view of strut 18 illustrating the increased thickness of the strut 18. In this embodiment, the portion 22 of increased thickness includes both the inner diameter and outer diameter of the strut.

FIG. 16 is a flat view of a portion of a stent 10 having struts 18 similar to those shown in FIGS. 14 and 15 wherein the portions 22 of increased thickness include both the inner diameter and the outer diameter of the strut.

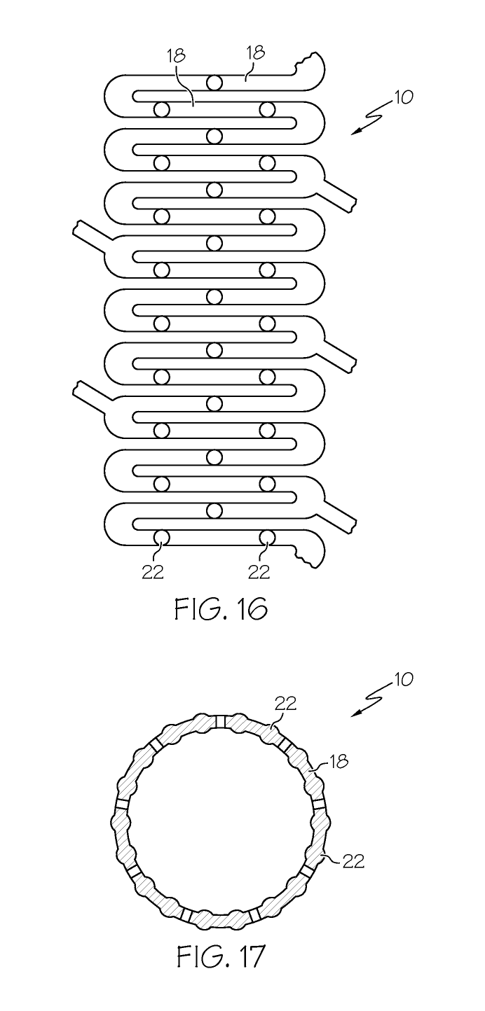

FIG. 17 is a radial cross-section of a stent 10 having struts 18 similar to those shown in FIGS. 14-16.

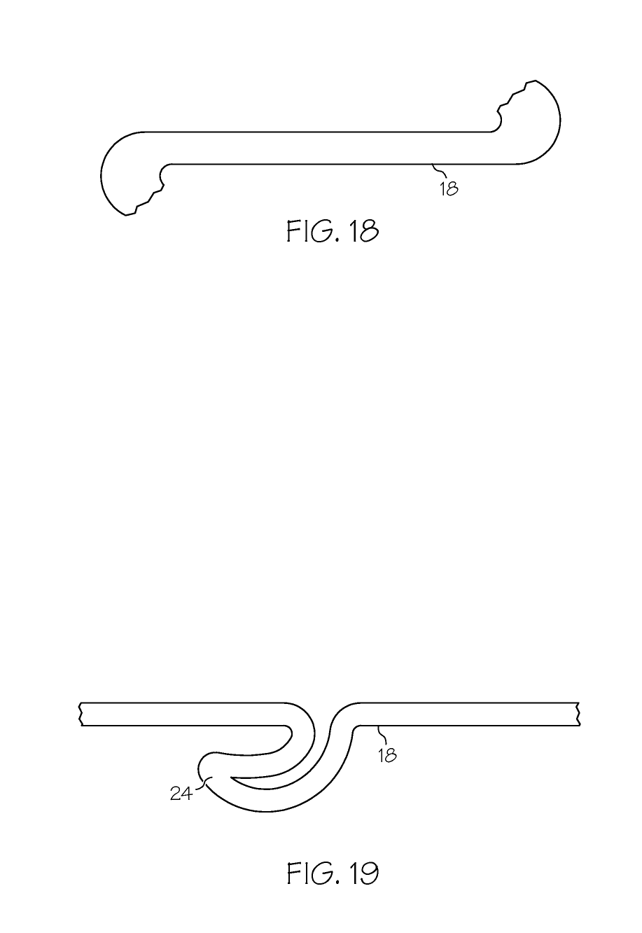

FIG. 18 is a side view of an elongated strut 18. In this embodiment, the elongated strut is folded along a portion 24 of the strut to form an area having increased radiopacity as shown in side view in FIG. 19.

A variety of methods can be employed in order to provide the stent with thicker/wider portions.

In some embodiment, portions of the stent are masked during electropolishing in order to limit the amount of metal removed from those portions of the stent. Using this technique, a strut pattern is laser machined or otherwise cut or etched into the stent preform. Post-laser finishing performed to remove laser affected metal and dross and to achieve finished stent mass and dimensions are not applied uniformly over the entire stent surface. The desired thicker stent portions can be masked. This may include the ends and/or middle serpentine bands, as well as any other pattern desired.

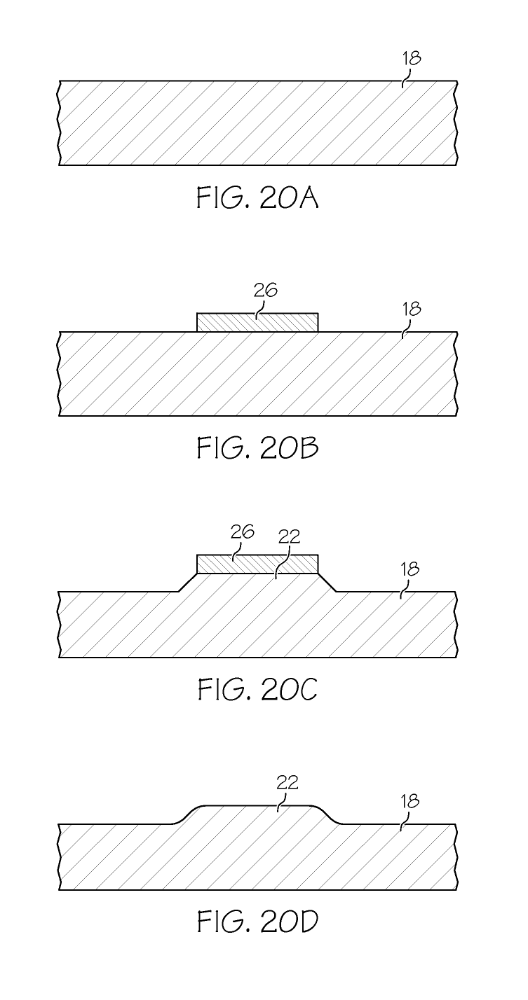

FIGS. 20a through 20d illustrate formation of the thicker portions using this technique. FIGS. 20a through 20d show a partial side view of a strut 18. In a first step, a strut pattern is formed in an iron stent preform via laser cutting as illustrated by FIG. 20a. A maskant 22 is then provided on a portion of each strut 18 wherein it is desirable to have increased wall thickness as shown in FIG. 20b. The masked strut 18 is then electropolished and material is removed from any unmasked surfaces as shown in FIG. 20c. The maskant is removed leaving a portion 22 of strut 18 with an increased thickness of at least about 0.0010″ relative to the stent wall immediately adjacent thereto. In this case, only the outer surface of the strut was masked so that increased wall thickness is seen only on the outer or abluminal strut surface and not on the inner or luminal strut surface.

In other embodiments, iron is deposited onto the stent preform after pattern formation but prior to final stent finishing steps. For example, in some embodiments, the iron is deposited after pattern formation via laser machining or other cutting or etching the pattern in the stent preform and after partial electropolishing. Deposition may be conducted via any suitable method such as laser deposition, electroplating or plasma deposition.

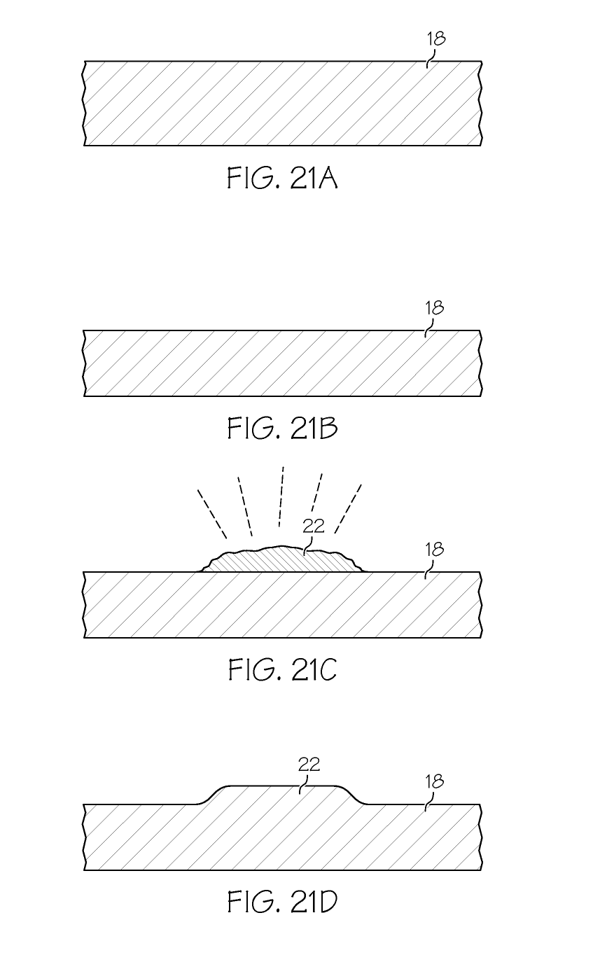

FIGS. 21a through 21d illustrate formation of thicker stent portions using metal deposition techniques. FIG. 21a is a side view of a stent strut 18 after laser cutting. The stent strut 18 is then partially electropolished represented by FIG. 21b. Focal deposition of iron is achieved via use of laser deposition, electroplating or plasma deposition as represented in FIG. 21c to form a portion 22 on the strut 18 of increased thickness. Final electropolishing is then conducted as shown in FIG. 21d leaving a portion 22 on the strut 18 of increased thickness of about 0.0010″ relative to the stent wall immediately adjacent thereto.

The description provided herein is not to be limited in scope by the specific embodiments described which are intended as single illustrations of individual aspects of certain embodiments. The methods, compositions and devices described herein can comprise any feature described herein either alone or in combination with any other feature(s) described herein. Indeed, various modifications, in addition to those shown and described herein, will become apparent to those skilled in the art from the foregoing description and accompanying drawings using no more than routine experimentation. Such modifications and equivalents are intended to fall within the scope of the appended claims.

All publications, patents and patent applications mentioned in this specification are herein incorporated by reference in their entirety into the specification to the same extent as if each individual publication, patent or patent application was specifically and individually indicated to be incorporated herein by reference. Citation or discussion of a reference herein shall not be construed as an admission that such is prior art.

1. A radially expandable stent, the stent made entirely of a bioabsorbable metal, the stent having an outer wall surface and an inner wall surface and a wall extending therebetween,
the wall characterized by a wall thickness as measure in a radial direction between the outer and inner wall surfaces,
the wall comprising a plurality of struts and connectors which are interconnected and which define openings in the wall,
each strut and each connector having two opposing sides extending between the outer and inner wall surfaces and having a length, and a width everywhere along its length,
the stent having a portion of increased radiopacity,
wherein the portion of increased radiopacity has one or more of the following characteristics:
i. the wall thickness of the stent in the portion of increased radiopacity exceeds the wall thickness of the wall immediately adjacent thereto by at least 0.0010″;
ii. the width of the stent in the portion of increased radiopacity exceeds the width of the stent immediately adjacent thereto by at least 0.0005″.

2. The radially expandable stent of claim 1 wherein said bioabsorbable metal composition is iron.

3. The radially expandable stent of claim 1 wherein the struts are arranged in serpentine bands and the connectors include first connectors which extend between adjacent serpentine bands and second connectors which extend between adjacent struts.

4. The stent of claim 1 comprising a plurality of portions of increased radiopacity, each of the plurality of portions having one or more of the following characteristics:
1. the wall thickness of the stent in the portion of increased radiopacity exceeds the wall thickness of the wall immediately adjacent thereto by at least 0.0010″;
2. the width of the stent in the portion of increased radiopacity exceeds the width of the stent immediately adjacent thereto by at least 0.0005″.

5. The radially expandable stent of claim 3 wherein at least some of the struts comprise said portion of increased radiopacity wherein the portion of increased radiopacity exceeds the wall thickness of the wall immediately adjacent thereto.

6. The radially expandable stent of claim 4 wherein at least some of the struts comprise said portion of increased radiopacity, the portion of increased radiopacity having a width that exceeds the width of the wall immediately adjacent thereto.

7. The radially expandable stent of claim 3 wherein the struts are interconnected by curved end portions, at least some of the curved end portions comprising said portion of increased radiopacity, the portion of increased radiopacity that exceeds the width of the wall immediately adjacent thereto.

8. The radially expandable stent of claim 3 wherein the struts are interconnected by curved end portions, at least some of the curved end portions comprising said portion of increased radiopacity, the portion of increased radiopacity that exceeds the thickness of the wall immediately adjacent thereto.

9. The radially expandable stent of claim 1 wherein the stent comprises a plurality of serpentine bands interconnected by connectors including a distal serpentine band, a proximal serpentine band and a middle serpentine band, each serpentine band comprising a plurality of parallel struts interconnected by curved end portions, and wherein at least one of said distal, proximal or middle serpentine band or combination thereof comprise at least one strut having the portion of increased radiopacity.

10. The radially expandable stent of claim 1 wherein the stent comprises a plurality of serpentine bands interconnected by connectors including a distal serpentine band, a proximal serpentine band and a middle serpentine band, each serpentine band comprising a plurality of parallel struts interconnected by curved end portions, and wherein at least some of said plurality of parallel struts of said distal, proximal or middle serpentine band or combination thereof comprise said portion of increased radiopacity.

11. The radially expandable stent of claim 1 wherein the stent comprises a plurality of serpentine bands interconnected by connectors including a distal serpentine band, a proximal serpentine band and a middle serpentine band, each serpentine band comprising a plurality of parallel struts interconnected by curved end portions, and wherein all of said plurality of parallel struts of said distal, proximal or middle serpentine band or combination thereof comprise said portion of increased radiopacity.

12. The radially expandable stent of claim 1 wherein the stent comprises a plurality of serpentine bands interconnected by connectors including a distal serpentine band, a proximal serpentine band and a middle serpentine band, each serpentine band comprising a plurality of parallel struts interconnected by curved end portions, and wherein all of said curved end portions of said distal, proximal or middle serpentine band or combination thereof comprise said portion of increased radiopacity.

13. A radially expandable stent, the radially expandable stent formed entirely from a bioabsorbable metal composition, the bioabsorbable metal composition is iron, the stent having an outer wall surface and an inner wall surface and a wall extending therebetween,
the wall characterized by a wall thickness as measure in a radial direction between the outer and inner wall surfaces,
the wall comprising a plurality of struts and connectors which are interconnected and which define openings in the wall,
each strut and each connector having two opposing sides extending between the outer and inner wall surfaces and having a length, and a width everywhere along its length,
the stent having a portion of increased radiopacity,
wherein the portion of increased radiopacity has one or more of the following characteristics:
i. the wall thickness of the stent in the portion of increased radiopacity exceeds the wall thickness of the wall immediately adjacent thereto by at least 0.0010″;
ii. the width of the stent in the portion of increased radiopacity exceeds the width of the stent immediately adjacent thereto by at least 0.0005″.

14. The radially expandable stent of claim 13 comprising a plurality of interconnected serpentine portions, each serpentine portion comprising a plurality of parallel struts interconnected by curved end portions, at least some of the struts, said at least some of said curved end portions or both comprising said portion of increased radiopacity.

15. A method of increasing the radiopacity of a bioabsorbable metal stent, the method including:
providing a tubular stent preform formed from a bioabsorbable metal;
cutting a strut pattern in the tubular stent preform, the strut pattern comprising parallel struts interconnected by curved end portions;
masking a portion of the tubular stent preform;
electropolishing the tubular stent preform; and
removing the masked portion;
wherein the masked portion comprises increased radiopacity and has one or more of the following characteristics:
i. the wall thickness of the stent in the masked portion of increased radiopacity exceeds the wall thickness of the wall immediately adjacent thereto by at least 0.0010″;
ii. the width of the stent in the masked portion of increased radiopacity exceeds the width of the stent immediately adjacent thereto by at least 0.0005″.

16. The method of claim 15 wherein the masked portion is on said parallel struts.

17. The method of claim 15 wherein the masked portion is on said curved end portions.

18. The method of claim 15 wherein said bioabsorbable metal stent is formed from iron.

19. A method of increasing the radiopacity of a bioabsorbable metal stent, the method including:
providing a tubular stent preform formed from a bioabsorbable metal;
cutting a strut pattern in the tubular stent preform;
partially electropolishing said tubular stent preform;
focally depositing said bioabsorbable metal on portions of the tubular stent preform to form portions of increased radiopacity; and
electropolishing said tubular stent preform;
wherein the portions of increased radiopacity and have one or more of the following characteristics:
i. the wall thickness of the stent in the masked portion of increased radiopacity exceeds the wall thickness of the wall immediately adjacent thereto by at least 0.0010″;
ii. the width of the stent in the masked portion of increased radiopacity exceeds the width of the stent immediately adjacent thereto by at least 0.0005″.

20. The method of claim 19 wherein said bioabsorbable metal is iron.

21. A radially expandable stent, the stent formed entirely from a bioabsorbable metal, the stent comprising a plurality of serpentine bands interconnected by connectors, each serpentine band comprising a plurality of struts characterized by a length and interconnected by curved end portions which define the length of the struts, wherein the struts are folded along a portion of the length, the folded portion comprising increased radiopacity.
